(19) United States
(12) Patent Application Publication
(10) Pub. No.: US 2025/0372250 A1
(43) Pub. Date: Dec. 4, 2025

(54) SYSTEM AND METHOD FOR DETECTING AGE-RELATED MACULAR DEGENERATION

(71) Applicant: MEDIOS TECHNOLOGIES PTE LTD, Singapore (SG)

(72) Inventors: Florian MicKael SAVOY, Icon (SG); Jun Kai TOH, Singapore (SG); Shao En Bryan ONG, Singapore (SG); Divya RAO PARTHASARATHY, Ellicott City, MD (US)

(21) Appl. No.: 18/848,325

(22) PCT Filed: Feb. 20, 2024
(86) PCT No.: PCT/IB2024/051604

§ 371 Date: Sep. 18, 2024

(30) Foreign Application Priority Data
Feb. 21, 2023 (IN) 202311011757

(51) Int. Cl. G16H 50/20; A61B 3/00; G16H 30/40

(52) U.S. Cl. CPC G16H 50/20; A61B 3/0025; G16H 30/40

(57) ABSTRACT

Approaches for detecting presence of AMD in an input eye include obtaining an input eye image, corresponding to the input eye. Once obtained, the input eye image undergoes a pre-processing step, including cropping. Thereafter, the input eye image is processed based on a view analysis model to select a macula centered view image. Then, the input eye image is processed based on a quality evaluation module to ascertain a quality of the input eye image. Once the input eye image is ascertained to be acceptable based on quality standards, the input eye image is processed based on an AMD detection model to obtain eye characteristic information to detect the presence of the AMD and perform a binary categorization of the input eye image as one of an AMD positive eye and an AMD negative eye.

### BACKGROUND

Eyes are the most delicate organ in the human body and to keep them unaffected from different visual disorders special care is required. One of the ways to provide special care is regular screening of the eyes to diagnose different visual disorders. Such screening or diagnosis may prevent odds, such as blurry vision, different color perception, and many more which may be caused by any developing eye disease, such as age-related macular degeneration (AMD). AMD is a leading cause of severe, irreversible vision impairment which usually develops in aged human beings. If not detected at its early stages, its effect may be amplified with each passing day. Generally, AMD is diagnosed by examining the retina of the eyes to detect the presence and features of tiny yellow deposits called drusen under the retina. However, none of the existing tests provide automatic detection of AMD with accurate results for large scale screening of population at minimal cost.

### BRIEF DESCRIPTION OF FIGURES

Systems and/or methods, is accordance with examples of the present subject matter are now described and with reference to the accompanying figures, in which:

FIG. 1A-1B illustrates a training system for training an Age-Related Macular Degeneration (AMD) detection model pipeline, as per an example;

FIG. 2 illustrates an AMD detection system for detecting presence of AMD in a subject's eye, as per one example;

FIG. 3 illustrates a method for training an AMD detection model pipeline, as per an example; and

FIGS. 4A-4B illustrate a method for detecting presence of AMD in the input eye image, based on a trained AMD detection model pipeline, as per an example.

### DETAILED DESCRIPTION

Eyes are the most used sensory organ among the five senses of the human body and eyes perceive most of the information about the world. Eye includes a retina at its back, which on illumination with light, cause the photoreceptors to turn the light into electrical signals. These electrical signals travel from the retina through an optic nerve to the brain for further processing. Such electric signals are then processed by the brain to create a visual feed or perception of surrounding objects which we see as images or videos.

An individual may suffer from different vision disorders. Examples of such vision disorders may include, but are not limited to, blurred vision (refractive errors), age-related macular degeneration, glaucoma, cataract, diabetic retinopathy, etc. One such visual disorder is age-related macular degeneration (AMD). AMD is a common eye condition and a leading cause of vision loss among people aged 50 and older. It causes damage to a macula, a small spot near the center of the retina and the part of the eye responsible for sharp, central vision, which is integral for activities where visual detail is of primary concern, such as reading, driving, and recognizing faces.

AMD is characterized by the presence of drusen, tiny yellow deposits under the retina, and changes in retinal pigment epithelium (RPE), which can lead to severe vision loss. Examples of some additional characteristic effects which may be caused by AMD on human eye include, but may not be limited to, presence of yellow droplets called drusen at back of the retina, retinal pigment epithelium (RPE) abnormalities such as hypopigmentation or hyperpigmentation, geographic atrophy of the RPE, choroidal neovascularization (exudative, wet), serous and/or hemorrhagic detachment of the sensory retina or RPE, subretinal and sub-RPE fibrovascular proliferation or disciform scar. AMD may be caused by a number of factors, e.g., aging, ethnicity, smoking, genetics, etc. The damage caused by AMD may not be reversed, but proper and timely diagnosis of AMD may prevent AMD from progressing.

The disease is typically diagnosed by examining the retina and detecting above described characteristic effects on the retina. Examples of conventional approaches include, but may not be limited to, Optical Coherence Tomography (OCT), fluorescein angiography, fundus photography, indocyanine Green, fundus autofluorescence, microperimetry, and adaptive optics. However, the process of diagnosing AMD can be complex and requires specialized medical practitioners and equipment, which may not be readily available in all healthcare settings, particularly in rural areas.

As may be understood, presence of such highly specialized medical practitioner and equipment is limited to tertiary level health care centers which are far away from the reach of rural population, which is highest in India. To perform screening of large population with minimal cost, there is a need for a system which performs automatic detection of AMD having an on-the-edge operable configuration to reduce cost and time of operation of such system.

Approaches for detecting presence of Age-related Macular Degeneration (AMD) in a subject's eye based on certain eye characteristics, are described. In one example, an input eye image which is to be screened for detecting AMD, is obtained. The input eye image may be an image of the eye of the subject which is under screening. Such input eye image may be either stored in a database repository or may be captured on real-time basis by a camera device. In another example, a set of input eye images may also be obtained for screening, e.g., one image for each eye (i.e., left eye and right eye) of the subject. In such a case, collective analysis of these images is used for determination of presence of AMD.

Once the input eye image is obtained, a pre-processing step on the input eye image is performed. In an example, the purpose of the pre-processing step is to make the input eye image compatible for further processing stages. For example, the pre-processing step is a cropping operation and via cropping operation the unnecessary parts of the input eye image are removed. Continuing further, the input eye image is processed to ascertain the view of the input eye image. Examples of various views possible for the input eye image include, but are not limited to, temporal view, nasal view, disc centered view, macula centered view, inferior view, and superior view. Further, in one example, in case of set of input eye images, the type of view of each of the input eye images may be ascertained.

Once the type of view of the input eye image is ascertained, a determination is performed to check whether the type of view is a macula centered view. For example, if the ascertained type of view is macula centered view, the input eye image may be utilized for further processing. On the other hand, if the input eye image is not of the macula centered view, the user or the subject may be prompted by displaying a visual indicator on a display device indicating instruction to capture another input eye image. Thereafter, the input eye image is processed to identify the presence of AMD in the subject's eye. However, some additional processing operations may also be performed before identifying the presence of AMD and one such operation is quality assessment.

In an example, the input eye image is processed to assess the quality of the input eye image to determine a quality score. Based on the quality score, if the quality score of the input eye image is determined to be greater than a threshold score, the input eye image may be passed onto perform identification of presence of AMD. On the other hand, if the determined quality score is less than the threshold score, the user or the subject may be prompted by displaying a visual indicator on the display to capture another input eye image.

Once the input eye image is ascertained to be acceptable based on quality standards, the same is further processed to identify eye characteristic information. In an example, the eye characteristic information corresponds to a plurality of eye image characteristics which individually or combinedly indicate either presence or absence of AMD in the subject's eye. Examples of eye image characteristics include, but are not limited to, size, area, color, and quantity of drusen at the back of retina, size, area, color and quantity of lesions at the level of retinal pigment epithelium (RPE), size, area, color, and quantity of drusen above the level of retinal pigment epithelium (RPE), other RPE changes, choroidal neovascularization or features suggestive of the same, geographic atrophy, disciform scar or a combination thereof.

Subsequently, based on the identified eye characteristic information, the AMD detection model detects presence of the AMD and performs binary categorization of the input eye image as one of an AMD positive (AMD eye) and an AMD negative (non-AMD eye). It may be noted that, although limited examples of eye image characteristics indicating presence or absence of AMD are described above, other such examples would still be within the scope of the present subject matter. In one example, the eye characteristic information may be used as a measurement parameter for ascertaining presence of AMD, as described subsequently.

It may be noted that the presence of AMD thus determined may be used to provide a further referral for treatment, or other intervention, as may be required. For example, a detection result may be generated which is indicative of a diagnosis of AMD. In addition to the result of detection of presence of AMD in the input eye image, a visualization output may be generated. In one example, the visualization output may be in the form of an activation map. The activation map thus obtained may indicate or highlight areas of abnormality in the input eye image which represents those areas or salient regions which lead to designation of input eye as referred AMD eye. These and other aspects have been discussed in further detail later in the present description.

It may be noted that the above-mentioned determinations involving view assessment, quality assessment, obtaining the eye characteristic information, detecting presence of AMD may involve a variety of models, such as the view analysis model, quality evaluation model, and the AMD detection model. In one example, each of the aforementioned models are machine learning based models. In an example, the machine learning model may be a deep learning model. Although having been described as unique or separate models, the view analysis model, quality evaluation model, and the AMD detection model may be implemented as an AMD detection model pipeline for the detection of AMD in the subject's eye. It may also be noted that an AMD detection system comprising the plurality of machine learning algorithms (such as view analysis model, quality evaluation model, and the AMD detection model) further includes an analysis engine which performs one or more intermediate functions, such as cropping operation on the input eye image, without deviating from the scope of the present subject matter.

The machine learning models within the AMD detection model pipeline may be trained based on a variety of training data. For example, the view analysis model may be trained based on training images having different views, e.g., images have temporal view, macula view, optic disc centered view, inferior view, superior view, and nasal view. Such training enables the view analysis model to identify the type of view of the input eye image. Similarly, the quality evaluation model may be trained based on a variety of training images having variety of resolution, contrast, clarity, or other such attributes. In a similar manner, the AMD detection model may be trained based on training images which are associated with AMD and the training images which are free of AMD, or not associated with AMD.

The AMD detection model may also be trained based on training eye characteristic information that may be obtained through clinical history, comprehensive eye examination and investigational modalities that include but not limited to optical coherence tomography, visual fields, intraocular pressure measurements, pachymetry etc. In an example, the AMD detection model includes two sub-models, i.e., a binary classification model which is trained to detect presence or absence of AMD and a categorical classification model which is trained to categorize eye image in various categories such as healthy eye, early AMD eye, intermediate AMD eye, and late AMD eye. In an example, the categorical classification model may be used only during training to supplement in the accuracy of detection of AMD by the binary classification model. Although the training has been described in the context of the view analysis model, quality evaluation model, and the AMD detection model, such similar training procedures may be performed for other models that may be implemented within the AMD detection model pipeline. Such processes would still fall within the scope of the present subject matter without limitation.

The present approaches overcome the above-mentioned technical advantages. For example, the above-mentioned approaches may be implemented in a single device for effective AMD screening. Since no specialized equipment or skill is required, a system implementing the present approaches is mobile, cost-effective, and accurate for the purposes of AMD detection. For example, an implementing system allows for screening without expert knowledge and is performable on portable retinal camera itself, while ensuring a desired and functional level of accuracy.

The explanation provided above and the examples that are discussed further in the current description are exemplary only. For instance, some of the examples may have been described in which only one image is considered or multiple images are considered, either in training or in inference stage. However, the current approaches may be adopted for other instances or situations as well without deviating from the scope of the present subject matter.

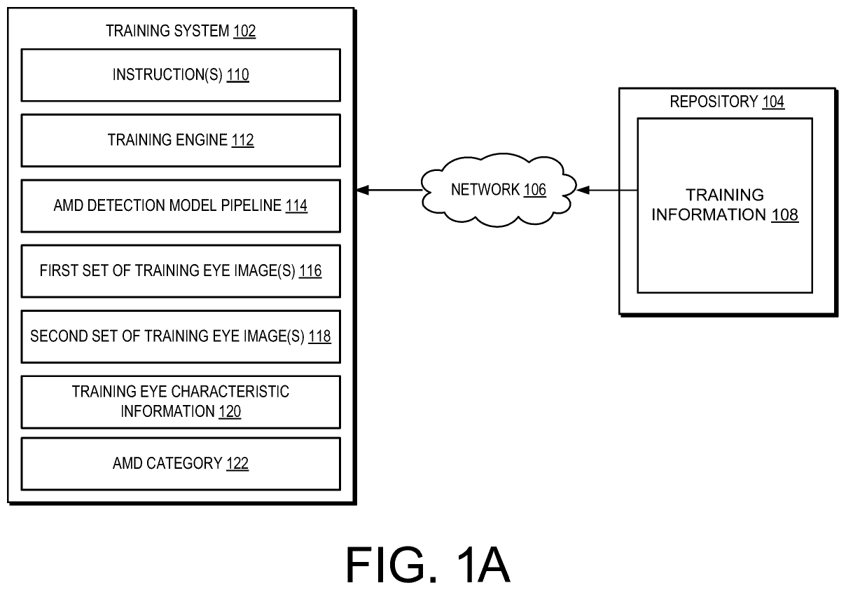

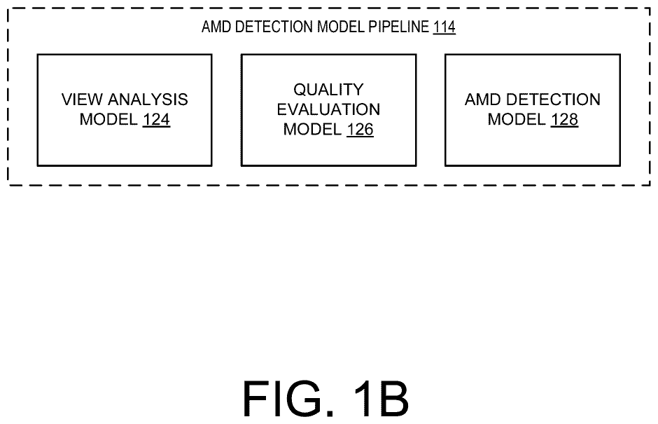

The manner in which the AMD detection model pipeline is trained and used for predicting presence of AMD in the input eye image is explained with respect to FIGS. 1-4. While aspects of described systems may be implemented in any number of different electronic devices, environments, and/or implementation, the examples are described in the context of the following example device(s). It may be noted that drawings of the present subject matter shown here are of illustrative purpose and are not to be construed as limiting the scope of the subject matter claimed.

FIG. 1 illustrates a training system 102 comprising a processor or memory (not shown), for training models present within an AMD detection model pipeline. In an example, the training system 102 (referred to as system 102) may be communicatively coupled to a repository 104 through a network 106. The repository 104 may further include training information 108. The training information 108 may include a first set of training eye images and a second set of training eye images captured from different sides and angles having different views. In an example, these pluralities of training eye images are those images which are captured previously while manual screening of the subject with corresponding AMD category annotated.

In an example, the first set of training eye images may include large dataset of eye images captured from a different camera or a normal pre-existing camera having normal quality. In an example, the first set of training eye images includes eye images collected while conducting surveys or any health program survey including images from different geographical regions of the world to provide a general learning to the AMD detection model pipeline. On the other hand, the second set of training eye images includes small dataset of images captured from a target camera having different image quality. In an example, the second set of training eye images include images from a specific geographic region to personalize the AMD detection model pipeline for that region corresponding particular ethnicity and genocity.

In another example, along with plurality of images, training information 108 may further include training eye characteristic information and corresponding AMD category for each of the plurality of training images representing severity of AMD in each of the training images. The training eye characteristic information corresponds to a plurality of training eye image characteristics. Examples of the plurality of training eye image characteristics include, but are not limited to, size, area, color, and quantity of drusen at the back of retina, size, area, color and quantity of lesions at the level of retinal pigment epithelium (RPE), size, area, color, and quantity of drusen above the level of retinal pigment epithelium (RPE), other RPE changes, choroidal neovascularization or features suggestive of the same, geographic atrophy, disciform scar or a combination thereof.

In another example, each of the set of training eye images, i.e., the first set of training eye images and the second set of training eye images, may include images in grouped manner in which each group includes images of single eye. The training information 108, although depicted as being obtained from a single repository, such as repository 104, may also be obtained from multiple other sources without deviating from the scope of the present subject matter. In such cases, each of such multiple repositories may be interconnected through a network, such as the network 106.

The network 106 may be a private network or a public network and may be implemented as a wired network, a wireless network, or a combination of a wired and wireless network. The network 106 may also include a collection of individual networks, interconnected with each other and functioning as a single large network, such as the Internet. Examples of such individual networks include, but are not limited to, Global System for Mobile Communication (GSM) network, Universal Mobile Telecommunications System (UMTS) network, Personal Communications Service (PCS) network, Time Division Multiple Access (TDMA) network, Code Division Multiple Access (CDMA) network, Next Generation Network (NGN), Public Switched Telephone Network (PSTN), Long Term Evolution (LTE), and Integrated Services Digital Network (ISDN).

The system 102 may further include instructions 110 and a training engine 112. In an example, the instructions 110 are fetched from a memory and executed by a processor included within the system 102. The training engine 112 may be implemented as a combination of hardware and programming, for example, programmable instructions to implement a variety of functionalities. In examples described herein, such combinations of hardware and programming may be implemented in several different ways. For example, the programming for the training engine 112 may be executable instructions, such as instructions 110. Such instructions may be stored on a non-transitory machine-readable storage medium which may be coupled either directly with the system 102 or indirectly (for example, through networked means). In an example, the training engine 112 may include a processing resource, for example, either a single processor or a combination of multiple processors, to execute such instructions. In the present examples, the non-transitory machine-readable storage medium may store instructions, such as instructions 110, that when executed by the processing resource, implement training engine 112. In other examples, the training engine 112 may be implemented as electronic circuitry.

The instructions 110, when executed by the processing resource, causes the training engine 112 to train a model pipeline, such as an AMD detection model pipeline 114 based on the training information 108. The instructions 110 may be executed by the processing resource for training the AMD detection model pipeline 114 based on the training information 108. The system 102 may further include a first set of training eye image(s) 116, a second set of training eye image(s) 118, training eye characteristic information 120, and an AMD category 122. In an example, the system 102 may obtain training information 108 corresponding to a plurality of eyes from the repository 104, and the information pertaining to that is stored as first set of training eye image(s) 116, second set of training eye image(s) 118, and AMD category 122 in the system 102 (the first set of training eye image(s) 116 and the second set of training eye image(s) 118 are combinedly referenced as training eye image(s) 116, 118 in further description of the invention).

As described previously, the AMD detection model pipeline 114 (referred to as detection model pipeline 114) may further include a plurality of machine learning models. An example of such machine learning models includes deep learning models. For the sake of explanation, the current approaches for detection of presence of AMD has been described with the different steps being performed using one or more deep learning models, as examples. Although the present examples have been described in relation to deep learning models, the aforementioned approaches may also be implemented using other machine-learning models. It may also be noted that any explanation provided in conjunction with deep learning models is applicable to other machine learning models, without limitations and without deviating from the scope of the present subject matter. Such examples have not been described for sake of brevity. The manner in which the training of the plurality of the models within the detection model pipeline 114 may be performed is further described in conjunction with FIG. 1B.

FIG. 1B depicts example deep learning models that may be implemented within the detection model pipeline 114. In one example, the detection model pipeline 114 may include a view analysis model 124, a quality evaluation model 126, and an AMD detection model 128. It may be noted that the detection model pipeline 114 may include other deep learning models (such as pre-processing model and processing model which are not shown in FIG. 1B) as well for implementing various other functions. It may also be the case that one or more models may be implemented so as to perform a combination of one or more functions. Such variations and combinations would still be examples of the present subject matter without limitations.

With respect to training the view analysis model 124, the training eye image(s) 116, 118 may be used wherein the training eye image(s) 116, 118 may include images having different views, e.g., temporal view, nasal view, disc centered view, macula centered view, inferior view, and superior view. For training the quality evaluation model 126, the training eye image(s) 116, 118 may include images having higher resolution, contrast, clarity, or other such attributes. The quality evaluation model 126 is trained to assess the quality of the input eye images so that the images having low quality may be discarded and only good quality images having higher quality are considered for further processing.

The AMD detection model 128 in turn may be trained based on training eye image(s) 116, 118. In an example, while training, the training engine 112 may process the AMD detection model 128 firstly based on the first set of training eye image(s) 116 which identify the training eye characteristic information 120 corresponding to the plurality of eye image characteristics within the first set of training eye image(s) 116. Examples of plurality of eye image characteristics include, but are not limited to, size, area, color, and quantity of drusen at the back of retina, size, area, color and quantity of lesions at the level of retinal pigment epithelium (RPE), size, area, color, and quantity of drusen above the level of retinal pigment epithelium (RPE), other RPE changes, choroidal neovascularization or features suggestive of the same, geographic atrophy, disciform scar or a combination of these features and many more. It may be noted that, above disclosed eye image characteristics are exemplary, distinct characteristics based on the type of images present in each set of training eye images may be used.

It may be noted that, once trained based on the first set of training eye image(s) 116, the AMD detection model 128 is capable of categorizing the eyes generally. However, since the first set of training eye image(s) 116 does not include images which are captured by target camera device of different quality, the AMD detection model 128 may miss the specific features of the eyes. For example, certain characteristics which only occur in the eyes of people of certain geographical regions. Therefore, such training made the AMD detection model 128 broadly predict the health category of the input eye image. Therefore, in order to train the AMD detection model 128 to predict the output more precisely, the AMD detection model 128 is further trained or fine-tuned using second set of training eye image(s) 118.

For example, while further training or fine-tuning, the training engine 112 may process the AMD detection model 128 based on the second set of training eye image(s) 118 which identify the training eye characteristic information 120 corresponding to the plurality of eye image characteristics within the second set of training eye image(s) 118. It may be noted that, once trained based on the second set of training eye image(s) 118, now, the AMD detection model 128 is capable of categorizing the eyes on personalized level. In addition to training images and corresponding characteristic information, the AMD detection model 128 may also be trained based on AMD category 122 associated with each of the training images. In an example, the AMD category 122 represents the state of corresponding training eye image. Therefore, based on the AMD category 122, i.e., either AMD positive or AMD negative, the training engine 112 accordingly identifies and learns eye characteristic information corresponding to the training eye images which are AMD positive.

In an example, the AMD detection model 128 includes two sub-models, i.e., a binary classification model which is trained to detect presence or absence of AMD and a categorical classification model which is trained to categorize eye image in various stages, such as healthy eye, early AMD eye, intermediate AMD eye, and late AMD eye. In an example, the categorical classification model may be used only during training to supplement in the accuracy of detection of AMD by the binary classification model.

As will be discussed subsequently, the view analysis model 124, the quality evaluation model 126, and the AMD detection model 128 when trained may be used to perform a variety of task either sequentially or concurrently based on which presence of AMD within a subject's eye may be ascertained. Further, as described above as well, the training of the view analysis model 124, the quality evaluation model 126, and the AMD detection model 128 may be performed in any order and may be performed at different instants. As may be understood, although one or more common training datasets may be used, the training of any one of the deep learning models in the detection model pipeline 114 is independent from the training of another model.

Once trained, the AMD detection model 128 may be used to determine or predict the presence of AMD in an input eye image. For example, the trained AMD detection model 128 identifies particular eye image characteristic information pertaining to the input eye image to determine an AMD category indicating presence of AMD in the input eye image. Once the AMD category of the input eye image is determined, the treatment appropriate for that category is suggested or determined to cure or prevent the enhancement of the visual disorder.

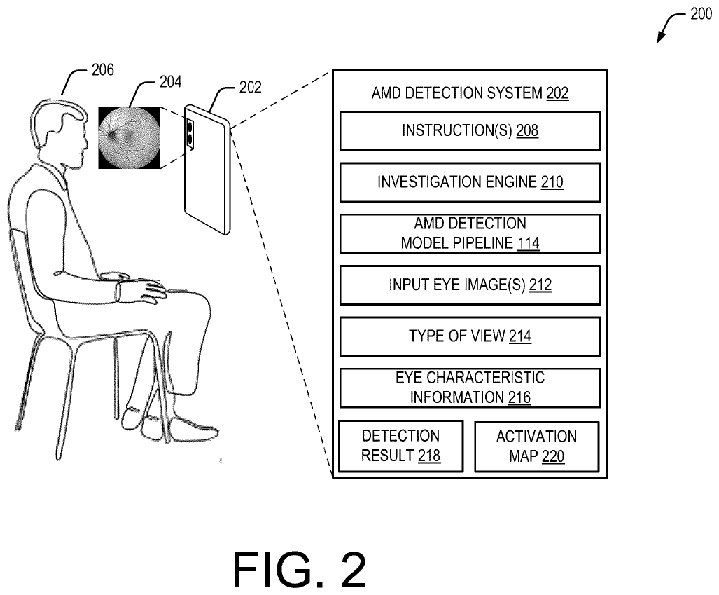

The manner in which the detection model pipeline may be used for detection of AMD within the subject's eye is further described in conjunction with FIG. 2.

FIG. 2 illustrates a clinical environment 200 with an Age-Related Macula Degeneration (AMD) detection system 202 for determining an AMD category to which an input eye image 204 of a subject 206 may pertain to. In an example, the AMD detection system 202 (referred to as system 202) is one of a mobile phone, tablet, or any other portable computing device. In an example, the portable computing device attached onto the system 202 is capable of capturing retinal images of the subject's eye. The input eye image 204 may be an image of an eye of the subject 206 who is under screening for the diagnosis of AMD. In an example, the input eye image 204 is a retinal image. In an example, the system 202 may analyze a plurality of eye image characteristics of the input eye image 204 based on the trained detection model pipeline 114.

Similar to the system 102, the system 202 may further include instructions 208 and an investigation engine 210. In an example, the instructions 208 are fetched from a memory and executed by a processor included within the system 202. The investigation engine 210 may be implemented as a combination of hardware and programming, for example, programmable instructions to implement a variety of functionalities. In examples described herein, such combinations of hardware and programming may be implemented in several different ways. For example, the programming for the investigation engine 210 may be executable instructions, such as instructions 208. Such instructions 208 may be stored on a non-transitory machine-readable storage medium which may be coupled either directly with the system 202 or indirectly (for example, through networked means). In an example, the investigation engine 210 may include a processing resource, for example, either a single processor or a combination of multiple processors, to execute such instructions. In the present examples, the non-transitory machine-readable storage medium may store instructions, such as instructions 208, that when executed by the processing resource, implement investigation engine 210. In other examples, the investigation engine 210 may be implemented as electronic circuitry.

In one example, the investigation engine 210 may utilize the trained detection model pipeline 114 to ascertain whether AMD is present within the subject's eye based on the processing of the input eye image 204 of the subject 206. It may be noted that the detection model pipeline 114 may be trained by way of the approach discussed in conjunction with FIGS. 1A-1B. As also described previously, the detection model pipeline 114 may further include the trained view analysis model 124, the quality evaluation model 126, and the AMD detection model 128.

The system 202 may further include an input eye image(s) 212, type of view 214, eye characteristic information 216, detection result 218, and activation map 220. It may be noted that the aforesaid data elements are generated by the investigation engine 210 using the detection model pipeline 114 and in response to the execution of the instruction(s) 208. These aspects and further details are discussed in the following paragraphs.

In operation, an input eye image, such as the input eye image 204 of an eye of the subject 206 who is under screening for the detection of presence of AMD, may be obtained. For example, the input eye image 204 may be captured through any image sensing sub-system that may be present within the system 202. In an example, the image sensing sub-system may be a retinal camera device which is either installed on the system 202 itself or may be removably integrated with the system 202. In another example, instead of having a single input eye image, a set of input eye images may be obtained in which image is present corresponding to each eye.

Once the input eye image 204 is obtained, the same is stored in the system 202 as input eye image(s) 212 (referred to as input eye image 212). Thereafter, the investigation engine 210 may perform a pre-processing step on the input eye image 212. The purpose of the pre-processing step is to make the input eye image 212 compatible for further processing stages and to remove unnecessary portions of the input eye image 212. Specifically, the pre-processing step includes a cropping operation and via cropping operation the unnecessary parts of the input eye image 212 are removed.

Continuing further, the input eye image 212 is processed to assess the view of the input eye image 212. In one example, investigation engine 210 may utilize the trained view analysis model 124 of the detection model pipeline 114 for ascertaining a type of view, such as type of view 214, of the input eye image 212. In an example, the trained view analysis model 124 assesses various features of the input eye image 212 to determine the view of the input eye image. Examples of various views possible for the input eye image 212 include, but are not limited to, temporal view, nasal view, disc centered view, macula centered view, inferior view, and superior view. Further, in one example, in case of set of input eye images, the type of view of each of the input eye images may be ascertained.

Once the type of view 214 of the input eye image 212 is ascertained, it is determined if the ascertained type of view 214 is macula centered view or not. If the ascertained type of view 214 is macula centered view, the input eye image 212 may be processed by the investigation engine 210 for further analysis. In an example, if the input eye image is not of the macula centered view, the user or the subject 206 may be prompted by displaying an indicator on a display of the system 202 to capture another input eye image 212 or may choose to proceed with the initially captured or obtained input eye image 212. In case where set of input eye images are obtained, among other images included in the set of input eye images, an image having macula centered view is selected for each eye of the subject 206 and is designated as the set of input eye images.

Continuing further, the input eye image 212 is processed to assess quality of the input eye image 212 using the trained detection model pipeline 114. In one example, the investigation engine 210 may utilize the trained quality evaluation model 126 of the detection model pipeline 114 for ascertaining a quality score for the input eye image 212. In an example, the quality score depicts the level of acceptance of the input eye image. For example, images having higher quality scores are accepted and images having lower quality score are discarded. In an example, high quality images are used as these images include clearer feature details.

Returning to the present example, once the quality score of the input eye image 212 is determined, if the quality score of the input eye image is greater than a threshold score, the input eye image 212 may be processed by the investigation engine 210 using the AMD detection model 128 to detect the presence of AMD in the subject's eye. In another example, if the quality score is less than the threshold score, the user or the subject 206 may be prompted by displaying an indicator on the display of the system 202 to capture another input eye image 212 or may choose to proceed with the initially captured or obtained input eye image 212. Both such examples are complimentary and as such have no impact on the scope of the present subject matter. It may be understood that ascertaining the quality of the input eye image 212 may rely on various features or attributes of the input eye image 212, as detected by the quality evaluation model 126. It may be noted that, in an example, the user or subject 206 may elect to proceed with subsequent process based on the input eye image 212 without assessing its quality, without deviating from the scope of the present subject matter.

The input eye image 212 (once determined as acceptable or as the case may be), may be further processed by the investigation engine 210 using the trained detection model pipeline 114 to identify eye characteristic information, such as eye characteristic information 216 of the input eye image 212. In one example, the investigation engine 210 may utilize the trained AMD detection model 128 of the detection model pipeline 114 to identify the eye characteristic information 216 of the input eye image 212. In an example, the eye characteristic information 216 corresponds to a plurality of eye image characteristics which individually or combinedly indicate either presence or absence of AMD in the subject's eye.

To this end, the investigation engine 210 may, using the AMD detection model 128, identify one or more eye image characteristics. Examples of the eye image characteristics include, but are not limited to, size, area, color, and quantity of drusen at the back of retina, size, area, color and quantity of lesions at the level of retinal pigment epithelium (RPE), size, area, color, and quantity of drusen above the level of retinal pigment epithelium (RPE), other RPE changes, choroidal neovascularization or features suggestive of the same, geographic atrophy, disciform scar or a combination thereof.

Based on the eye characteristic information 216 thus determined using the trained AMD detection model 128, the investigation engine 210 may further process the eye characteristic information 216 based on the AMD detection model 128 to determine a detection result, such as detection result 218 for the input eye image 212 corresponding to the subject's eye. In an example, the detection results 218 represent absence or presence of AMD within the input eye image 212 of the subject eye. In another example, in case of multiple input eye images, the investigation engine 210 may determine the detection result 218 representing absence or presence of AMD in the subject's eye as a whole by considering input eye images corresponding to each eye of the subject 206. Based on the detection result 218, the investigation engine 210 may categorize the input eye image 212 as one of the AMD positive eye and the AMD negative eye.

It may be noted that the detection results 218 thus determined may be used to provide a further referral for treatment, or other intervention, as may be required. For example, the detection results 218 may be indicative of a diagnosis of AMD. Based on the state represented by the detection result 218, appropriate action may be taken. Although explained as being obtained by processing above-described examples of eye image characteristic included in the eye characteristic information 216, the detection of presence of AMD may be performed by considering any other eye image characteristics without deviating from the scope of the present subject matter. Such examples would still fall within the scope of the present subject matter, without any limitation.

In furtherance to this, the investigation engine 210 may also generate the activation map 220 to be displayed on the display device of the system 202. In an example, the activation map 220 depicts salient regions within the input eye image 212 which triggered the detection of AMD within the input eye image 212 of the subject 206. Further, the displayed activation map may also be used by medical practitioner to identify the regions which have caused the disease.

In another example, the system 202 may be communicatively coupled to a central computing server through a network (not shown in FIG. 2). The network may be a private network or a public network and may be implemented as a wired network, a wireless network, or a combination of a wired and wireless network, and may be similar to the network 106 (as depicted in FIG. 1A). All the above disclosed steps which may be performed by the investigation engine 210 of the system 202, may be implemented or performed by the central computing server on behalf of the system 202 to reduce computing load on edge of the network.

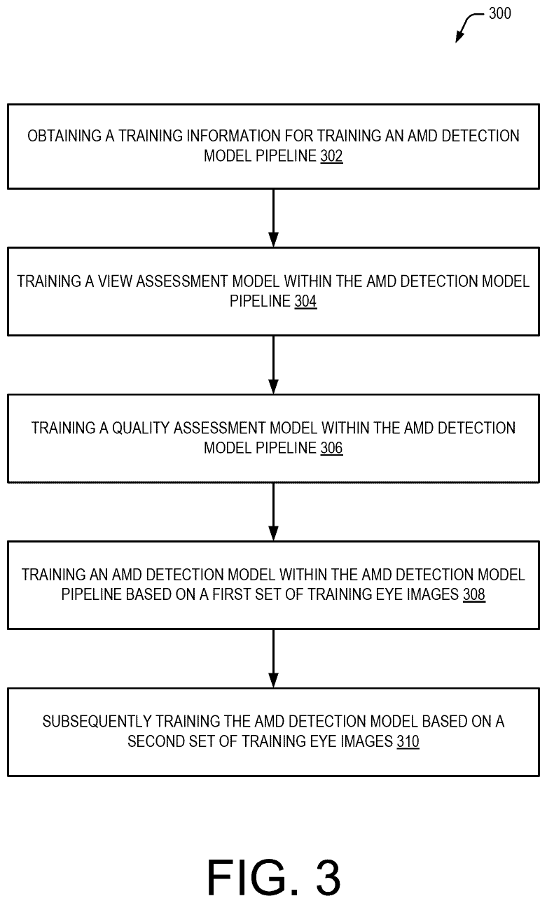

FIG. 3 illustrates example method 300 for training a detection model pipeline, in accordance with examples of the present subject matter. The order in which the above-mentioned method is described is not intended to be construed as a limitation, and some of the described method blocks may be combined in a different order to implement the method, or alternative method.

Furthermore, the above-mentioned method may be implemented in suitable hardware, computer-readable instructions, or combination thereof. The steps of such method may be performed by either a system under the instruction of machine executable instructions stored on a non-transitory computer readable medium or by dedicated hardware circuits, microcontrollers, or logic circuits. For example, the method may be performed by a training system, such as system 102. In an implementation, the method may be performed under an “as a service” delivery, where the system 102, operated by a provider, receives programmable code. Herein, some examples are also intended to cover non-transitory computer readable medium, for example, digital data storage media, which are computer readable and encode computer-executable instructions, where said instructions perform some or all the steps of the above-mentioned method.

In an example, the method 300 may be implemented by the system 102 for training several deep learning models based on a training information, such as training information 108. At block 302, training information for training a detection model pipeline may be obtained. For example, the system 102 may obtain training information 108. The training information 108 may be obtained from a repository, such as the repository 104. The training information 108 may include a first set of training eye images and a second set of training eye images captured from different sides and angles having different views. In an example, these pluralities of training eye images are those images which are captured previously while manual screening of the subject with corresponding AMD category annotated.

In an example, the first set of training eye image(s) may include large dataset of eye images captured from a different camera or a normal pre-existing camera having normal quality. In an example, the first set of training eye image(s) includes eye images collected while conducting surveys or any health program survey including images from different geographical regions of the world to provide a general learning to a detection model pipeline, such as the AMD detection model pipeline 114. On the other hand, the second set of training eye images include small dataset of images captured from a target camera having different image quality. In an example, the second set of training eye images include images from a specific geographic region to personalize the AMD detection model pipeline 114 for that region.

In another example, along with the plurality of images, the training information 108 may include training eye characteristic information and corresponding AMD category for each of the plurality of training images representing severity of AMD in each of the training images. The training eye characteristic information corresponds to a plurality of training eye image characteristics. Examples of plurality of training eye image characteristic include, but are not limited to, size, area, color, and quantity of drusen at the back of retina, size, area, color and quantity of lesions at the level of retinal pigment epithelium (RPE), size, area, color, and quantity of drusen above the level of retinal pigment epithelium (RPE), other RPE changes, choroidal neovascularization or features suggestive of the same, geographic atrophy, disciform scar or a combination thereof.

In another example, each of the set of training eye images, i.e., first set of training eye images and the second set of training eye images, may include images in grouped manner in which each group includes images of single eye.

At block 304, a view analysis model which is present within a detection model pipeline may be trained. In one example, the training engine 112 may train the view analysis model 124 using the training eye image(s) 116, 118 wherein the training eye image(s) 116, 118 may include images having different views, e.g., temporal view, nasal view, disc centered view, macula centered view, inferior view, and superior view. The view analysis model 124 is thus trained to ascertain the view of the input eye image.

At block 306, a quality evaluation model of a detection model pipeline may be trained. For example, the training engine 112 of the system 102 may train the quality evaluation model 126 based on the training eye image(s) 116, 118 which may include images having higher resolution, distinct varieties of contrast, clarity, or other such attributes. The quality evaluation model 126 is trained to assess the quality of the input eye images so that the images having low quality may be discarded and only good quality images having higher quality are considered for further processing.

At block 308, an AMD detection model of a detection model pipeline may be trained. For example, while training the AMD detection model 128, the training engine 112 may process the AMD detection model 128 firstly based on the first set of training eye image(s) 116 which identify the training eye characteristic information 120 corresponding to the plurality of eye image characteristics within the first set of training eye image(s) 116. Examples of plurality of eye image characteristics include, but are not limited to, size, area, color, and quantity of drusen at the back of retina, size, area, color and quantity of lesions at the level of retinal pigment epithelium (RPE), size, area, color, and quantity of drusen above the level of retinal pigment epithelium (RPE), other RPE changes, choroidal neovascularization or features suggestive of the same, geographic atrophy, disciform scar or a combination of these features and many more. It may be noted that, above disclosed eye image characteristics are exemplary, distinct characteristics based on the type of images present in each set of training eye images may be used.

It may be noted that, once trained based on the first set of training eye image(s) 116, the AMD detection model 128 is capable of categorizing the eyes generally. However, since the first set of training eye image(s) 116 does not include images which are captured by target camera device of different quality, the AMD detection model 128 may miss the specific features of the eyes. For example, certain characteristics which only occur in the eyes of people of certain geographical regions. Therefore, such training made the AMD detection model 128 broadly predict the health category of the input eye image. Therefore, in order to train the AMD detection model 128 to predict the output more precisely, the AMD detection model 128 is further trained or fine-tuned using second set of training eye image(s) 118.

At block 310, the AMD detection model is subsequently trained based on the second set of training eye images. For example, while further training or fine-tuning, the training engine 112 may process the AMD detection model 128 based on the second set of training eye image(s) 118 which identify the training eye characteristic information 120 corresponding to the plurality of eye image characteristics within the second set of training eye image(s) 118. It may be noted that, once trained based on the second set of training eye image(s) 118, now, the AMD detection model 128 is capable of categorizing the eyes on personalized level. In addition to training images and corresponding characteristic information, the AMD detection model 128 may also be trained based on AMD category 122 associated with each of the training images. In an example, the AMD category 122 represents the state of corresponding training eye image. Therefore, based on the AMD category 122, i.e., either AMD positive or AMD negative, the training engine 112 accordingly identifies and learns eye characteristic information corresponding to the training eye images which are AMD positive.

In an example, the AMD detection model 128 includes two sub-models, i.e., a binary classification model which is trained to detect presence or absence of AMD and a categorical classification model which is trained to categorize eye image in various stages, such as healthy eye, early AMD eye, intermediate AMD eye, and late AMD eye. In an example, the categorical classification model may be used only during training to supplement in the accuracy of detection of AMD by the binary classification model.

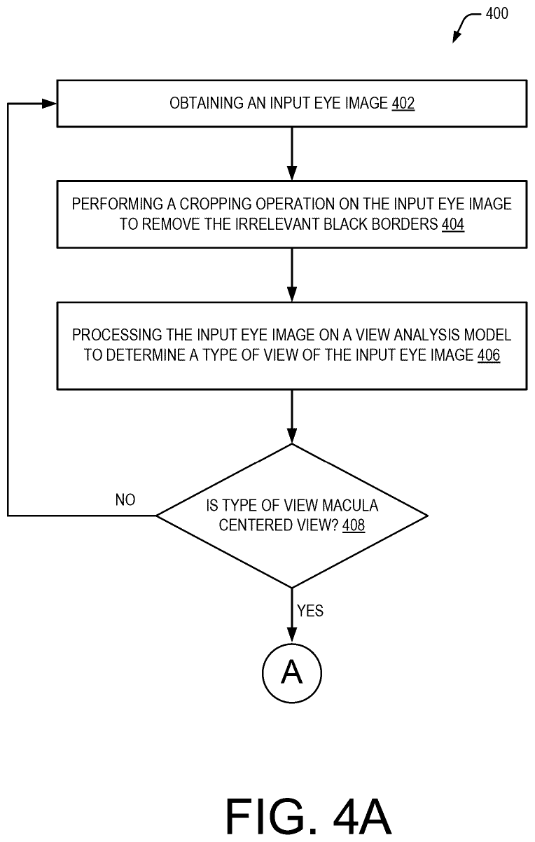

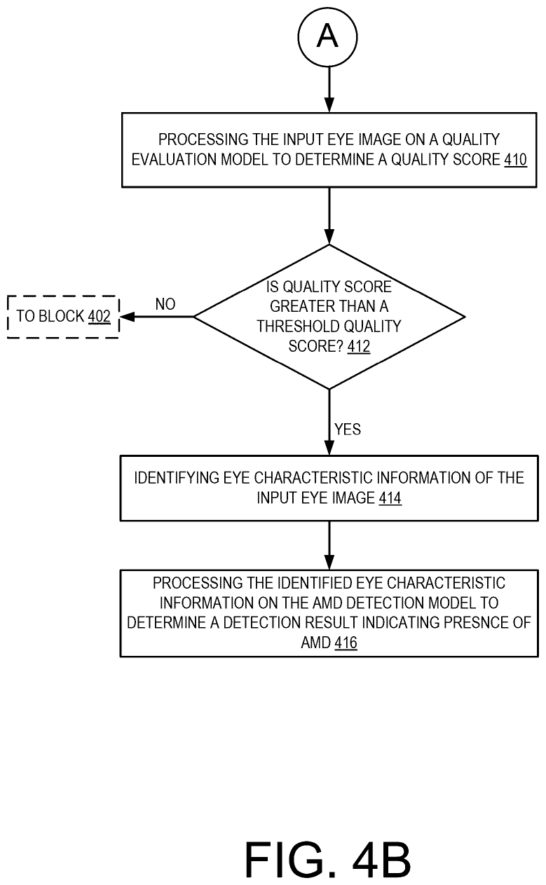

In an example, once trained, the detection model pipeline 114 may be utilized for categorizing an input eye image as one of an AMD positive and an AMD negative. The method steps involved in categorizing the input eye image as one of AMD positive and AMD negative are further described in conjunction with FIG. 4A-4B.

FIGS. 4A-4B illustrate example method 400 for categorizing an input image under one of AMD positive (AMD eye) and AMD negative (non-AMD eye) category. Similar to FIG. 3, the order in which the above-mentioned method is described is not intended to be construed as a limitation, and some of the described method blocks may be combined in a different order to implement the method, or alternative method. Based on the present approaches as described in the context of the example method 400, the eye image characteristics of an input eye image is analyzed based on the trained detection model pipeline 114.

Further, the above-mentioned method 400 may be implemented in a suitable hardware, computer-readable instructions, or combination thereof. The steps of such method may be performed by either a system under the instruction of machine executable instructions stored on a non-transitory computer readable medium or by dedicated hardware circuits, microcontrollers, or logic circuits. For example, the method may be performed by an AMD detection system, such as system 202. In an implementation, the method may be performed under an “as a service” delivery model, where the system 202, operated by a provider, receives programmable code. Herein, some examples are also intended to cover non-transitory computer readable medium, for example, digital data storage media, which are computer readable and encode computer-executable instructions, where said instructions perform some or all the steps of the above-mentioned method.

At block 402, an input eye image is obtained. For example, the input eye image 204 may be captured through any image sensing sub-system that may be present within the system 202. In an example, the image sensing sub-system may be a retinal camera device which is either installed on the system 202 itself or may be removably integrated with the system 202. In another example, instead of having a single input eye image, a set of input eye images may be obtained in which image is present corresponding to each eye.

At block 404, a cropping operation may be performed on the input eye image thus obtained. For example, once the input eye image 204 (or input eye image 212) is obtained, the investigation engine 210 may perform a pre-processing step on the input eye image 212. The purpose of the pre-processing step is to make the input eye image 212 compatible for further processing stages and to remove unnecessary portions of the input eye image 212. Specifically, the pre-processing step includes the cropping operation and via the cropping operation the unnecessary parts of the input eye image 212 are removed.

At block 406, a type of view of the input eye image is determined by processing the input eye image. For example, the investigation engine 210 may utilize the trained view analysis model 124 of the detection model pipeline 114 for ascertaining a type of view, such as type of view 214, of the input eye image 212. In an example, the trained view analysis model 124 assesses various features of the input eye image 212 to determine the view of the input eye image. Examples of various views possible for the input eye image 212 include, but are not limited to, temporal view, nasal view, disc centered view, macula centered view, inferior view, and superior view. Further, in one example, in case of set of input eye images, the type of view of each of the input eye images may be ascertained.

At block 408, a determination is made to ascertain whether the type of view of the input eye image is macula centered view or not. For example, if the ascertained type of view 214 is macula centered view, the input eye image 212 may be further processed by the investigation engine 210 using the detection model pipeline 114 (‘Yes’ path from block 408), as will be described in later steps. If, however, the input eye image 212 is not of macula centered view, the subject 206 may be prompted by displaying an indicator on a display of the system 202 to capture or obtain another input eye image 212 (‘No’ path from block 408) or may choose to proceed with the initially captured or obtained input eye image 212. In case of set of input eye images obtained, among other images included in the set of input eye images, an image having macula centered view is selected for each eye of the subject 206 and is designated as the set of input eye images.

At block 410, the input eye image may be further processed based on a quality evaluation model to determine its acceptability. For example, investigation engine 210 may utilize the trained quality evaluation model 126 of the detection model pipeline 114 for ascertaining a quality score for the input eye image 212. In an example, the quality score depicts the level of acceptance of the input eye image. For example, images having higher quality score are accepted and images having lower quality score are discarded. In an example, high quality images [symbol] as these images include feature details clearer.

At block 412, a determination is made whether the determined quality score of the input eye image is acceptable or not. For example, if the quality score of the input eye image is determined to be greater than a threshold score, the input eye image 212 may be processed by the investigation engine 210 using the AMD detection model to detect the presence of AMD in the subject's eye (‘Yes’ path from block 412). If, however, the determined quality score is less than the threshold score, the user or the subject 206 may be prompted by displaying an indicator on the display of the system 202 to capture another input eye image 212 (‘No’ path from block 412) or may choose to proceed with the initially captured or obtained input eye image 212. Both such examples are complimentary and as such have no impact on the scope of the present subject matter. It may be understood that ascertaining the quality of the input eye image 212 may rely on various features or attributes of the input eye image 212, as detected by the quality evaluation model 126. It may be noted that, in an example, the user or subject 206 may elect to proceed with subsequent process based on the input eye image 212 without assessing its quality, without deviating from the scope of the present subject matter.

At block 414, the input eye image may be further processed based on the AMD detection model to identify the eye characteristic information of the input eye image. For example, the investigation engine 210 may utilize the trained AMD detection model 128 of the detection model pipeline 114 to identify the eye characteristic information 216 of the input eye image 212. In an example, the eye characteristic information 216 corresponds to a plurality of eye image characteristics which individually or combinedly indicate either presence or absence of AMD in the subject's eye.

To this end, the investigation engine 210 may, using the AMD detection model 128, identify one or more eye image characteristics. Examples of the eye image characteristics include, but are not limited to, size, area, color, and quantity of drusen at the back of retina, size, area, color and quantity of lesions at the level of retinal pigment epithelium (RPE), size, area, color, and quantity of drusen above the level of retinal pigment epithelium (RPE), other RPE changes, choroidal neovascularization or features suggestive of the same, geographic atrophy, disciform scar or a combination thereof.

At block 416, the determined eye characteristic information may be further processed based on the AMD detection model to determine a detection result indicating the presence or absence of AMD in the subject's eye. For example, the investigation engine 210 may further process the eye characteristic information 216 based on the AMD detection model 128 to determine the detection result 218 for the input eye image 212 corresponding to the subject's eye. In an example, the detection results 218 represents absence or presence of AMD within the input eye image 212 of the subject eye. In another example, in case of multiple input eye images, the investigation engine 210 may determine the detection result 218 representing absence or presence of AMD in the subject's eye as a whole by considering input eye images corresponding to each eye of the subject 206. Based on the detection result 218, the investigation engine 210 categorize the input eye image 212 as one of the AMD positive eye and the AMD negative eye.

It may be noted that the detection results 218 thus determined may be used to provide a further referral for treatment, or other intervention, as may be required. For example, the detection results 218 may be indicative of a diagnosis of AMD. Based on the state represented by the detection result 218, appropriate action may be taken. Although explained as being obtained by processing above-described examples of eye image characteristic included in the eye characteristic information 216, the detection of presence of AMD may be performed by considering any other eye image characteristics without deviating from the scope of the present subject matter. Such examples would still fall within the scope of the present subject matter, without any limitation.

In furtherance to this, the investigation engine 210 may also generate the activation map 220 to be displayed on the display device of the system 202. In an example, the activation map 220 depicts salient regions within the input eye image 212 which triggered the detection of AMD within the input eye image 212 of the subject 206. Further, the displayed activation map may also be further used by medical practitioner to identify the regions which have caused the disease.

Although examples for the present disclosure have been described in language specific to structural features and/or methods, it is to be understood that the appended claims are not necessarily limited to the specific features or methods described. Rather, the specific features and methods are disclosed and explained as examples of the present disclosure.

1. A system comprising:
a processor; and
an investigation engine, coupled to the processor, for:
obtaining an input eye image corresponding to a subject's eye, wherein the subject is under screening for detecting presence of Age-Related Macular Degeneration (AMD);
using an AMD detection model pipeline, wherein the AMD detection model pipeline is trained based on a training information comprising training images associated with the AMD and training eye characteristic information corresponding to a plurality of training eye image characteristics of each of the training images, wherein on feeding the input eye image, wherein on using the AMD detection model, the AMD detection model is for:
determining a type of view of the input eye image;
identifying an eye characteristic information from the input eye image corresponding to a plurality of input eye image characteristics on determining the type of view as a macula centered view; and
generating a detection result indicating presence of AMD within the subject's eye based on the eye characteristic information.

2. The system as claimed in claim 1, wherein the investigation engine is for:
performing a cropping operation on the input eye image to remove irrelevant portions of the input eye image.

3. The system as claimed in claim 1, wherein the AMD detection pipeline is for:
discarding the input eye image when the type of view of the input eye image is other than the macula centered view.

4. The system as claimed in claim 1, wherein the investigation engine is for:
obtaining plurality of input eye images, wherein the plurality of input eye images comprises a first set of eye images corresponding to the left eye and a second set of eye images corresponding to the right eye, wherein the each of the plurality of input eye images corresponds to different views of the subject's eye;
feeding the plurality of input eye images into the AMD detection model pipeline, wherein on feeding the AMD detection model pipeline is for:
determining the view of each of the plurality of input eye images;
designating an image having macula centered view for each eye as input eye image based on the determined view;
identifying the eye characteristic information corresponding to the designated image for each eye; and
generating the detection result indicating presence of AMD within the subject's eye based on the eye characteristic information of each eye combinedly.

5. The system as claimed in claim 1, wherein the AMD detection model pipeline comprises a plurality of deep learning models selected from a group comprising a view analysis model, a quality evaluation model, and an AMD detection model.

6. The system as claimed in claim 1, wherein the investigation engine is for:
performing a quality evaluation of the input eye image to generate a quality score;
determining the type of view of the input eye image on determining the quality score to be greater than a threshold quality score; or
discarding the input eye image on determining the quality score to be less than the threshold quality score; and
prompting a user to obtain or capture a fresh input eye image.

7. The system as claimed in claim 1, wherein the plurality of input eye image characteristics comprises size, area, color, and quantity of drusen at the back of retina, size, area, color and quantity of lesions at the level of retinal pigment epithelium (RPE), size, area, color, and quantity of drusen above the level of retinal pigment epithelium (RPE), other RPE changes, choroidal neovascularization or features suggestive of the same, geographic atrophy, disciform scar or a combination thereof.

8. The system as claimed in claim 1, wherein the investigation engine is further for:
generating an activation map highlighting areas of abnormality in the input eye image, wherein the highlighted areas have led to designation of the input eye as AMD positive eye.

9. A method comprising:
obtaining a training information comprising a first set of training eye images and a second set of training eye images, wherein each of the training eye images are associated with corresponding training eye image characteristics and Age-Related macula degeneration (AMD) category;
training an AMD detection model pipeline based on the first set of training eye images, corresponding training eye characteristic information and associated AMD category, wherein training eye characteristic information corresponds to a plurality of training eye image characteristics; and
subsequently training the AMD detection model pipeline based on the second of training eye images, corresponding training eye characteristic information and associated AMD category.

10. The method as claimed in claim 9, wherein the plurality of training eye image characteristics comprises size, area, color, and quantity of drusen at the back of retina, size, area, color and quantity of lesions at the level of retinal pigment epithelium (RPE), size, area, color, and quantity of drusen above the level of retinal pigment epithelium (RPE), other RPE changes, choroidal neovascularization or features suggestive of the same, geographic atrophy, disciform scar or a combination thereof.

11. The method as claimed in claim 9, wherein the first set of training eye images comprises a large dataset of training eye images captured from a camera device having pre-existing image capturing capability, wherein the AMD detection model pipeline when trained using the first set of training eye images is to learn eye characteristic information for a general population having AMD.

12. The method as claimed in claim 9, wherein the second set of training eye images comprises a small dataset of images from a specific geographical region captured from a target camera device having specific image capturing capability, wherein when trained using the second set of training eye images, the AMD detection pipeline model is personalized for a specific population having AMD.

13. The method as claimed in claim 9, wherein the AMD detection model pipeline comprises a plurality of deep learning models selected from a group comprising a view analysis model, a quality evaluation model, and an AMD detection model.

14. The method as claimed in claim 9, wherein the method comprises assessing, by the trained AMD detection model pipeline, quality of the input eye image to discard images having unacceptable quality.

15. The method as claimed in claim 9, wherein the method comprises determining, by the trained AMD detection model pipeline, type of view of the input image to accept only images having macula centered view, wherein the input eye image have one of a temporal view, nasal view, disc centered view, macula centered view, inferior view, and superior view.
